(19) United States
(12) Patent Application Publication
(10) Pub. No.: US 2026/0248691 A1
(43) Pub. Date: Aug. 27, 2026

(54) DEVICES FOR USE IN NASAL TREATMENT

(71) Applicant: Sa'id DAKWAR, Maalot Tarshiha (IL)

(72) Inventor: Sa'id DAKWAR, Maalot Tarshiha (IL)

(21) Appl. No.: 19/163,165

(22) PCT Filed: Mar. 7, 2024
(86) PCT No.: PCT/IL2024/050252

§ 371 Date: Sep. 8, 2025

(30) Foreign Application Priority Data
Mar. 8, 2023 (IL) 301205

(51) Int. Cl. A61J 17/00; A61M 11/00; A61M 21/00

(52) U.S. Cl. CPC A61J 17/10; A61J 17/001; A61M 11/006; A61M 21/00; A61M 2021/0016; A61M 2210/0618

(57) ABSTRACT

Infant nasal treatment pacifier kit including pacifier and nasal spray add-on device for nasal treatment. The pacifier including: mouth plate having distal and proximal side, a nipple on proximal side; and add-on receiving portion on the distal side to detachably receive the add-on device. The add-on device, for use with a nasal spray bottle having an outlet and containing fluid for ejecting into infant's nostrils using the add-on device, including: a proximal pacifier engaging portion detachably attachable to an add-on accommodating portion of the pacifier; upper portion including two nasal ports on two sides of a vertical symmetry plane and having length to be insertable into the infant's nostrils; and distal bottle engaging portion having inlet to securely and detachably receive the nasal spray bottle outlet; the inlet in fluid communication with the nasal ports, to guide ejected fluid from the nasal spray bottle through nasal ports.

### TECHNOLOGICAL FIELD

The presently disclosed subject matter relates to the field of devices such as pacifiers and/or add-on devices for pacifiers that provide treatment substances to infants.

### BACKGROUND ART

A pacifier having a means for providing nasal treatment is disclosed in the following patents:

U.S. Pat. No. 10,751,256B1 discloses a system including a pacifier, an aroma device secured to the pacifier, and an aroma scented material. The pacifier includes a solid body with a front surface and a back surface; and a nipple secured to and extending from the front surface. The aroma device includes a housing secured to and protruding from the back surface of the body, the housing forming an inner cavity. The aroma scented material is removably secured within the inner cavity.

US20180117275A1 discloses an aroma emitting pacifier for an infant that gives off a predetermined scent by evaporation of volatile essential oils or similar substances. The aroma emitting pacifier is constructed to prevent evaporation of the essential oil before activation, so that the nipple does not become contaminated with a flavor the infant may find unpalatable, and so that the oil is conserved until it is desired to administer it to the infant.

WO 2018127464A1 discloses a pacifier for generating an aerosol, comprising a nipple, and an atomizing unit which is configured to atomize a liquid.

WO2011137905A1 discloses a soother provided with a fluid guide comprising one or more conduits for supplying e.g., oxygen to the nostrils of an infant. The fluid guide is manufactured as a single piece to ensure a simple design. The soother can be assembled from three or four parts which make the manufacturing of the soother simple. The soother may be designed so that parts of the soother are releasably connectable. Thereby, the soother may be disassembled after use so that efficient cleaning is possible. Accordingly, the cleaned parts can be assembled possibly in combination with new parts and other part types. The design of the fluid guide ensures convenient supply of e.g., oxygen to the nostrils and convenient connection of oxygen hoses.

There are also known pacifiers for providing oxygen through nostrils of an infant. Examples of these can be found in US2012/0247469, U.S. Pat. No. 5,375,593A, and US20040040556A1.

U.S. Pat. No. 7,753,886B2 discloses a medicine dispensing pacifier that has a centered nipple upon a guard and a reservoir communicating through the guard to the nipple. The reservoir, opposite the guard, has a cap, and upon removing it, a caregiver places medicine into the reservoir. Then the caregiver returns the cap to seal the reservoir. The infant then suckles the nipple, ingesting the medicine drawn from the reservoir. Alternately, the pacifier includes an air permeable membrane, a flexible cap, for dispensing medicine under pressure through a nipple into an infant that has difficulty taking medicine. The infant then suckles upon the nipple and the caregiver presses upon the cap raising the flow of medicine through the nipple into the mouth of the infant. The cap augments the natural suckling of the infant with pressure and without spilling medicine.

### GENERAL DESCRIPTION

Parents and caregivers tasked with administering treatment substances to infants often face difficulties, as infants can exhibit uncooperative behavior and may refuse treatment altogether. These difficulties can be compounded when a treatment requires prolonged access to the nostrils and/or mouth of an infant, as infants' tendency to squirm and move around can make it even more difficult to administer the treatment effectively. Such difficulties can be frustrating for parents and caregivers, and in some instances may present as an insurmountable obstacle, ultimately resulting in the infant not receiving necessary treatment. Leaving conditions such as nasal congestion, fever, or bacterial infection untreated can lead to prolonged suffering and in some cases hospitalization of the infant. It is important to provide innovative solutions that can improve the ability of parents and caregivers to administer treatment substances to infants.

In accordance with different aspects of the presently disclosed subject matter there is provided at least one add-on device which can be used for delivering treatment substances to an infant and which can constitute a part of a kit therefor, which can further comprise at least one pacifier to which the at least one add-on device can be attached, and/or other accessories. One type of such add-on device, according to one aspect of the presently disclosed subject matter, can be a nasal spray add-on device configured for use with a nasal spray bottle having an outlet and containing a fluid to be ejected into nostrils of an infant using said add-on device. The fluid can be a saline solution, which is commonly used to reduce nasal congestion. In this case the add-on device can comprise an upper portion comprising two nasal ports insertable into the nostrils of an infant, and a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.

If intended for the attachment to a pacifier, the nasal spray add-on device can have a pacifier engaging portion. The pacifier engaging portion can be specifically designed to be detachably attachable to a corresponding add-on receiving portion of the pacifier.

Another type of the add-on devices which can also be attachable to the same or a different pacifier, can be an aromatherapy add-on device. The aromatherapy add-on device can be detachably fixable to a pacifier, and can comprise at least one recess for an olfactory-active substance to be deposited therewithin and slowly release the add-on the olfactory-active substance. According to one aspect of the presently disclosed subject matter, there is provided a pacifier kit for performing nasal treatment to an infant, the kit comprising at least a pacifier, and at least one nasal spray add-on device directed for a nasal treatment, said pacifier comprising:
a mouth plate having a distal and a proximal side,
a nipple formed on the proximal side, and
an add-on receiving portion formed on the distal side and configured to detachably receive said nasal spray add-on device;

wherein said nasal spray add-on device is configured for use with a nasal spray bottle having an outlet and containing a fluid to be ejected into the nostrils of an infant using said nasal spray add-on device, said nasal spray add-on device comprising;
a proximal pacifier engaging portion having such shape and dimensions as to be detachably attachable to said add-on accommodating portion of the pacifier,
an upper portion comprising two nasal ports, said nasal ports being so disposed on two sides of a vertical plane of symmetry and having such a length as to allow them to be insertable into the nostrils of an infant when said pacifier is in use, and
a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.

According to another aspect of the presently disclosed subject matter, at least one of the above add-on devices or any other add-on devices and a pacifier to which it can be detachably attachable can constitute a pacifier kit for performing nasal treatment to an infant. The pacifier kit can comprise more that one add-on device. When the pacifier kit comprises at least two add-on devices, the at least two add-on devices can be directed for different nasal treatments, and can be interchangeably attachable to the pacifier. The pacifier of such kit can particularly comprise a mouth plate having a distal and a proximal side; a nipple formed on the proximal side; and an add-on receiving portion formed on the distal side and configured to interchangeably and fixedly receive an add-on device. The pacifier kit can additionally include external devices or materials that are to be used in conjunction with the at least one add-on devices. For example, the pacifier kit may include a nasal spray bottle to be used with a nasal spray add-on device, or olfactory materials to be used with an aromatherapy add-on device. The pacifier kit may also include an oral medicine delivery pacifier, to broaden the scope of treatment the pacifier kit can provide, for the convenience of a caregiver.

The oral medicine delivery pacifier may comprise; a nipple, a shield having a first and a second side, and a fluid compartment. The nipple can be being formed on said first side of said shield and have an interior lumen. The fluid compartment can be detachably attachable to the distal side of said shield, or optionally, uniformly formed therewith, and have a reservoir in fluid communication with the interior channel of the nipple. An infant sucking on the nipple can draw fluid from the fluid compartment and ingest the fluid. The fluid compartment can comprise an outer flexible cap which can be pressed inwards into the fluid compartment by a parent or caregiver to force liquid from the reservoir through the interior channel of the nipple.

According to a further aspect of the presently disclosed subject matter, there is provided a method for preparing a nasal spray bottle having an outlet for use in administration of a nasal spray into the nostrils of an infant, comprising; providing a nasal spray add-on device having two nasal ports and a bottle engaging portion, and connecting said outlet of said nasal spray bottle to said nasal spray add-on device so as to enable ejecting nasal spray from said nasal spray bottle into said two nasal ports. The nasal spray bottle for use with the nasal spray add-on device can further comprise an actuator attached to the outlet thereof, and the method can further comprise removing the actuator, so that the spring-loaded outlet can be connected to the nasal spray add-on device.

According to a further aspect of the of the presently disclosed subject matter, there is provided a method for preparing a nasal spray bottle having an outlet for use in administration of a nasal spray into the nostrils of an infant, comprising
providing a nasal spray add-on device having two nasal ports and a bottle engaging portion;
providing a pacifier having an add-on accommodating portion;
attaching said nasal spray add-on device to said add-on accommodating portion of the pacifier; and
connecting said outlet of said nasal spray bottle to said nasal spray add-on device so as to enable ejecting nasal spray from said nasal spray bottle into said two nasal ports.

In all the above aspects of the presently disclosed subject matter, the nasal spray add-on device can comprise at least the following:
an upper portion comprising two nasal ports, said nasal ports being so disposed on two sides of a vertical plane of symmetry and having such a length as to allow them to be insertable into the nostrils of an infant; and
a distal bottle engaging portion having an inlet configured to securely and detachably receive the outlet of the nasal spray bottle, the inlet being in fluid communication with the nasal ports, so as to guide ejected fluid from the nasal spray bottle through the two nasal ports.

According to one aspect of the presently disclosed subject matter, there is provided a nasal spray add-on device configured for use with a nasal spray bottle having an outlet and containing a fluid to be ejected into nostrils of an infant using said nasal spray add-on device, said nasal spray add-on device further configured for use with a pacifier comprising an add-on receiving portion formed on a distal side thereof and configured to receive said nasal spray add-on device, said nasal spray add-on device comprising;
a proximal pacifier engaging portion having such shape and dimensions as to be detachably attachable to said add-on accommodating portion of said pacifier,
an upper portion comprising two nasal ports, said nasal ports being so disposed on two sides of a vertical plane of symmetry and having such a length as to allow them to be insertable into the nostrils of an infant, and
a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.

Furthermore, in all the above aspects of the presently disclosed subject matter, the nasal spray add-on device can comprise at least one or more of the following features in any combination thereof:
the distal bottle engaging portion can comprise a stream splitting element comprising an inlet constituting the inlet of the add-on device and two outlets in fluid communication with the two nasal ports;
   the stream splitting element can have a Y shape defined by a stem and two branches; said inlet can be constituted by the stem, and the two outlets can be constituted by the two branches;
   the distal bottle engaging portion can have an interior, and the stream splitting element can be located at least partially within the interior;
the distal bottle engaging portion can comprise at least one alignment ridge shaped to align said bottle outlet with said inlet;
   when the distal bottle engaging portion has an interior, the at least one alignment ridges can be at least partially disposed within said interior;
   the proximal pacifier engaging portion can comprise a central aperture configured to receive the add-on accommodating portion of a pacifier.

The spray bottle for use with the nasal spray add-on device can comprise a pump, optionally provided with a spring-loaded mechanism. In this case, the nasal spray add-on device can further comprise at least one fingerhold configured to be at least partially gripped by the user when actuating said spring-loaded mechanism. Alternatively, the spray bottle can be of a kind other than a spring-loaded injector and the nasal spray add-on device can still have at least one fingerhold to be at least partially gripped by the user when actuating the device. In both cases, the fingerhold can be constituted by a gripping ridge on an exterior surface of the distal bottle engaging portion.

If intended for the use with a pacifier, the method for preparing a nasal spray bottle having an outlet for use in administration of a nasal spray into the nostrils of an infant can further comprise attaching the pacifier engaging portion of the nasal spray add-on device to the add-on engaging portion of the pacifier.

Reverting to the aromatherapy add-on device, which can constitute a part of a pacifier kit according to one of the above aspects of the presently disclosed subject matter, such device can comprise at least one or more of the following features in any combination thereof;
a pacifier engaging portion can have such shape and dimensions as to be detachably fixable to the add-on receiving portion of a pacifier;
at least one recess for an olfactory-active substance to be deposited therewithin, the at least once recess can comprise an absorbent material configured to retain and slowly release the add-on the olfactory-active substance, the absorbent material being open to the exterior of the device along at least the majority of the recess;
   the at least one recess can comprise at least one channel, and the at least one channel can be an annular channel extending along an outer surface of the add-on device;
   the absorbent material of the aromatherapy add-on device can be wood;
a central pacifier engaging aperture having said pacifier engaging portion and being configured to be received within the add-on receiving portion of the pacifier;
a proximal face and a and distal face, wherein the proximal face can be configured to face the pacifier and the distal face can have said recess;
the aromatherapy add-on device can be ring-shaped;
the aromatherapy add-on device can comprise an absorbing element made of said absorbent material and comprising said at least one recess, and an adaptor comprising said pacifier engaging portion and configured for securely holding said absorbing element.

Further embodiments of aspects described above are listed below.
1. A pacifier kit for performing nasal treatment to an infant, said kit comprising at least a pacifier, and at least one nasal spray add-on device directed for a nasal treatment, said pacifier comprising:
   a mouth plate having a distal and a proximal side,
   a nipple formed on the proximal side, and
   an add-on receiving portion formed on the distal side and configured to detachably receive said nasal spray add-on device;
wherein said nasal spray add-on device is configured for use with a nasal spray bottle having an outlet and containing a fluid to be ejected into the nostrils of an infant using said nasal spray add-on device, said nasal spray add-on device comprising;
   a proximal pacifier engaging portion having such shape and dimensions as to be detachably attachable to said add-on accommodating portion of the pacifier,
   an upper portion comprising two nasal ports, said nasal ports being so disposed on two sides of a vertical plane of symmetry and having such a length as to allow them to be insertable into the nostrils of an infant when said pacifier is in use, and
   a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.
2. A pacifier kit according to embodiment 1, wherein said spray bottle is in the form of a spring-loaded injector comprising a spring-loaded mechanism.
3. A pacifier kit according to embodiment 2, wherein said nasal spray add-on device further comprises at least one fingerhold configured to be at least partially gripped by the user when actuating said spring-loaded mechanism.
4. A pacifier kit according to embodiment 3 wherein said distal bottle engaging portion comprises a stream splitting element comprising said inlet and two outlets in fluid communication with said two nasal ports.
5. A pacifier kit according to embodiment 4 wherein said distal bottle engaging portion has an interior and the stream splitting element is located at least partially within said interior.
6. A pacifier kit according to embodiment 5 wherein said stream splitting element has a Y shape defined by a stem and two branches, and said inlet is constituted by said stem and the two outlets are constituted by said branches.
7. A pacifier kit according to any one of embodiments 2-6 wherein said distal portion of said nasal spray add-on device comprises alignment ridges shaped to align said bottle outlet with said inlet.
8. A pacifier kit according to embodiment 7 when dependent on embodiment 5 wherein said alignment ridges are at least partially disposed within said interior.
9. A pacifier kit according to embodiment 3, wherein said fingerhold is constituted by a ridge on an exterior of the distal bottle engaging portion.
10. A pacifier kit according to embodiment 1 wherein said proximal pacifier engaging portion comprises a central aperture configured to receive the add-on accommodating portion of a pacifier.
11. A pacifier kit according to any one of the preceding embodiments, further comprising at least one additional device directed for a different nasal treatment.
12. A pacifier kit according to embodiment 11, wherein said additional device is constituted by an aromatherapy add-on device, said aromatherapy add-on device comprising; at least one recess for an olfactory-active substance to be deposited therewithin, said at least once recess comprising an absorbent material configured to retain and slowly release the add-on the olfactory-active substance, said absorbent material being open to the exterior of the device along at least the majority of the recess; and a pacifier engaging portion having such shape and dimensions as to be detachably fixable to the add-on receiving portion.
13. A pacifier kit according to embodiment 12 wherein said at least one recess of the aromatherapy add-on device comprises at least one channel.
14. A pacifier kit according to embodiment 13 wherein said at least one channel of the at least one recess of the aromatherapy add-on device is an annular channel extending along an outer surface of the add-on device.
15. A pacifier kit according to embodiment 12 wherein said absorbent material of the aromatherapy add-on device is wood.
16. A pacifier kit according to embodiment 12 wherein said aromatherapy add-on device further comprises a central pacifier engaging aperture having said pacifier engaging portion and configured to be received within the add-on receiving portion of the pacifier.
17. A pacifier kit according to embodiment 12, wherein said aromatherapy add-on device has proximal and distal faces, said proximal face being configured to face the pacifier and said distal face having said recess.
18. A pacifier kit according to embodiment 12, wherein said aromatherapy add-on device is ring shaped.
19. A pacifier kit according to embodiment 12, wherein said aromatherapy add-on device comprises an absorbing element made of said absorbing material and comprising said recess, and an adaptor comprising said pacifier engaging portion and configured for securely holding said absorbing element.
20. A nasal spray add-on device configured for use with a nasal spray bottle having an outlet and containing a fluid to be ejected into nostrils of an infant using said nasal spray add-on device, said nasal spray add-on device further configured for use with a pacifier comprising an add-on receiving portion formed on a distal side thereof and configured to receive said nasal spray add-on device, said nasal spray add-on device comprising;
   a proximal pacifier engaging portion having such shape and dimensions as to be detachably attachable to said add-on accommodating portion of said pacifier,
   an upper portion comprising two nasal ports, said nasal ports being so disposed on two sides of a vertical plane of symmetry and having such a length as to allow them to be insertable into the nostrils of an infant, and
   a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.
21. A nasal spray add-on device according to embodiment 20, wherein said spray bottle comprises a pump, optionally provided with a spring-loaded mechanism.
22. A nasal spray add-on device according to embodiment 21, wherein said nasal spray add-on device further comprises at least one fingerhold configured to be at least partially gripped by the user when actuating said spring-loaded mechanism.
23. A nasal spray add-on device according to embodiment 20 wherein said distal bottle engaging portion comprises a stream splitting element comprising said inlet and two outlets in fluid communication with said two nasal ports.
24. A nasal spray add-on device according to embodiment 23 wherein said distal bottle engaging portion has an interior and the stream splitting element is located at least partially within said interior.
25. A nasal spray add-on device according to embodiment 24 wherein said stream splitting element has a Y shape defined by a stem and two branches, and said inlet is constituted by said stem and the two outlets are constituted by said branches.
26. A nasal spray add-on device according to any one of embodiments 20-25, wherein said distal portion of said nasal spray add-on device comprises at least one alignment ridge shaped to align said bottle outlet with said inlet.
27. A nasal spray add-on device according to embodiment 26 when dependent on embodiment
24 wherein said at least one alignment ridge is at least partially disposed within said interior.
28. A nasal spray add-on device according to embodiment 22, wherein said fingerhold is constituted by at least one gripping ridge on an exterior surface of the distal bottle engaging portion.
29. A nasal spray add-on device according to embodiment 28, wherein said proximal pacifier engaging portion comprises a central aperture configured to receive the add-on accommodating portion of a pacifier.
30. A method for preparing a nasal spray bottle having an outlet for use in administration of a nasal spray into the nostrils of an infant, comprising
   providing a nasal spray add-on device having two nasal ports and a bottle engaging portion;
   providing a pacifier having an add-on accommodating portion;
   attaching said nasal spray add-on device to said add-on accommodating portion of the pacifier; and
   connecting said outlet of said nasal spray bottle to said nasal spray add-on device so as to enable ejecting nasal spray from said nasal spray bottle into said two nasal ports.
31. A method according to embodiment 30, wherein the nasal spray add-on device further comprises;
   an upper portion comprising two nasal ports, said nasal ports being on two sides of a vertical plane of symmetry, so as to be insertable into the nostrils of an infant, and
   a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.
32. A method according to embodiment 31, wherein said spray bottle comprises a pump, optionally provided with a spring-loaded mechanism.
33. A method according to embodiment 32, wherein said ejecting nasal spray from said nasal spay bottle comprises actuating said spring-loaded mechanism.
34. A method according embodiment 33, wherein said nasal spray add-on device further comprises at least one fingerhold configured to be at least partially gripped by the user when actuating said spring-loaded mechanism.
35. A method according to embodiment 31, wherein said distal bottle engaging portion comprises a stream splitting element comprising said inlet and two outlets in fluid communication with said two nasal ports.
36. A method according to embodiment 35, wherein said distal bottle engaging portion has an interior and the stream splitting element is located at least partially within said interior.
37. A method according to embodiment 35 wherein said stream splitting element has a Y shape defined by a stem and two branches, and said inlet is constituted by said stem and the two outlets are constituted by said branches.
38. A method according to any one of embodiments 31-37, wherein said distal portion of said nasal spray add-on device comprises at least one alignment ridge shaped to align said bottle outlet with said inlet.
39. A method according to embodiment 38, when dependent on embodiment 36 wherein said at least one alignment ridge is at least partially disposed within said interior.
40. A method according to embodiment 34, wherein said fingerhold is constituted by a gripping ridge on an exterior surface of the distal bottle engaging portion.
41. A method according to embodiment 32, wherein the nasal spray bottle further comprises an actuator attached to the outlet thereof, and the method further comprises removing said actuator, so that the outlet can be connected to the nasal spray device.
42. A method according to embodiment 41, wherein said proximal pacifier engaging portion comprises a central aperture configured to receive the add-on accommodating portion of a pacifier.
43. A pacifier kit for performing nasal treatment to an infant, said kit comprising at least a pacifier, and at least one device directed for a nasal treatment, said pacifier comprising:
   a mouth plate having a distal and a proximal side,
   a nipple formed on the proximal side, and
   an add-on receiving portion formed on the distal side and configured to interchangeably and fixedly receive one of said two devices directed for different nasal treatments, said devices optionally being an aromatherapy add-on device and a nasal spray add-on device,
said pacifier kit further comprising an oral medicine delivery pacifier, said oral medicine delivery pacifier comprising a nipple, a shield having a first and a second side, and a fluid compartment;
   said nipple being formed on said first side of said shield, and said nipple having an interior lumen;
   said fluid compartment being detachably attachable to the distal side of said shield, or optionally, uniformly formed therewith, and having a reservoir in fluid communication with the interior channel of the nipple, said fluid compartment comprising an outer flexible cap which can be pressed inwards into the fluid compartment to force liquid from the reservoir through the interior channel of the nipple.
44. A pacifier kit according to embodiment 43, wherein said nasal spray add-on device is configured for use with a nasal spray bottle having an outlet and containing a fluid to be ejected into the nostrils of an infant using said nasal spray add-on device, said nasal spray add-on device comprising;
   a proximal pacifier engaging portion having such shape and dimensions as to be detachably attachable to said add-on accommodating portion of the pacifier,
   an upper portion comprising two nasal ports, said nasal ports being so disposed on two sides of a vertical plane of symmetry and having such a length as to allow them to be insertable into the nostrils of an infant when said pacifier is in use, and
   a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.
45. A pacifier kit according to embodiment 44, wherein said spray bottle is in the form of a spring-loaded injector comprising a spring-loaded mechanism.
46. A pacifier kit according to embodiment 45, wherein said nasal spray add-on device further comprises at least one fingerhold configured to be at least partially gripped by the user when actuating said spring-loaded mechanism.
47. A pacifier kit according to embodiment 44 wherein said distal bottle engaging portion comprises a stream splitting element comprising said inlet and two outlets in fluid communication with said two nasal ports.
48. A pacifier kit according to embodiment 47 wherein said distal bottle engaging portion has an interior and the stream splitting element is located at least partially within said interior.
49. A pacifier kit according to embodiment 74 wherein said stream splitting element has a Y shape defined by a stem and two branches, and said inlet is constituted by said stem and the two outlets are constituted by said branches.
50. A pacifier kit according to any one of embodiments 44-49 wherein said distal portion of said nasal spray add-on device comprises alignment ridges shaped to align said bottle outlet with said inlet.
51. A pacifier kit according to embodiment 50 when dependent on embodiment 48 wherein said alignment ridges are at least partially disposed within said interior.
52. A pacifier kit according to embodiment 46, wherein said fingerhold is constituted by a ridge on an exterior of the distal bottle engaging portion.
53. A pacifier kit according to embodiment 44 wherein said proximal pacifier engaging portion comprises a central aperture configured to receive the add-on accommodating portion of a pacifier.
54. A pacifier kit according to any one of the preceding embodiments, further comprising at least one additional device directed for a different nasal treatment.
55. A pacifier kit according to any one of embodiments 43-54, wherein said additional device is constituted by an aromatherapy add-on device, said aromatherapy add-on device comprising;
   at least one recess for an olfactory-active substance to be deposited therewithin, said at least once recess comprising an absorbent material configured to retain and slowly release the add-on the olfactory-active substance, said absorbent material being open to the exterior of the device along at least the majority of the recess; and
   a pacifier engaging portion having such shape and dimensions as to detachably fixable to the add-on receiving portion.
56. A pacifier kit according to embodiment 55 wherein said at least one recess of the aromatherapy add-on device comprises at least one channel.
57. A pacifier kit according to embodiment 56 wherein said at least one channel of the at least one recess of the aromatherapy add-on device is an annular channel extending along an outer surface of the add-on device.
58. A pacifier kit according to embodiment 55 wherein said absorbent material of the aromatherapy add-on device is wood.
59. A pacifier kit according to embodiment 55 wherein said aromatherapy add-on device further comprises a central pacifier engaging aperture having said pacifier engaging portion and configured to be received within the add-on receiving portion of the pacifier.
60. A pacifier kit according to embodiment 55, wherein said aromatherapy add-on device has proximal and distal faces, said proximal face being configured to face the pacifier and said distal face having said recess.
61. A pacifier kit according to embodiment 55, wherein said aromatherapy add-on device is ring shaped.
62. A pacifier kit according to embodiment 55, wherein said aromatherapy add-on device comprises an absorbing element made of said absorbing material and comprising said recess, and an adaptor comprising said pacifier engaging portion and configured for securely holding said absorbing element.
63. A pacifier kit for performing nasal treatments to an infant, said kit comprising at least a pacifier, and at least two devices directed for different nasal treatments, said pacifier comprising:
   a mouth plate having a distal and a proximal side,
   a nipple formed on the proximal side, and
   an add-on receiving portion formed on the distal side and configured to interchangeably and fixedly receive one of said two devices directed for different nasal treatments, said devices optionally being an aromatherapy add-on device and a nasal spray add-on device.
64. A pacifier kit according to embodiment 63, wherein said nasal spray add-on device is configured for use with a nasal spray bottle having an outlet and containing a fluid to be ejected into the nostrils of an infant using said nasal spray add-on device, said nasal spray add-on device comprising;
   a proximal pacifier engaging portion having such shape and dimensions as to be detachably attachable to said add-on accommodating portion of the pacifier,
   an upper portion comprising two nasal ports, said nasal ports being so disposed on two sides of a vertical plane of symmetry and having such a length as to allow them to be insertable into the nostrils of an infant when said pacifier is in use, and
   a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.
65. A pacifier kit according to embodiment 64, wherein said spray bottle is in the form of a spring-loaded injector comprising a spring-loaded mechanism.
66. A pacifier kit according to embodiment 65, wherein said nasal spray add-on device further comprises at least one fingerhold configured to be at least partially gripped by the user when actuating said spring-loaded mechanism.
67. A pacifier kit according to embodiment 64 wherein said distal bottle engaging portion comprises a stream splitting element comprising said inlet and two outlets in fluid communication with said two nasal ports.
68. A pacifier kit according to embodiment 67 wherein said distal bottle engaging portion has an interior and the stream splitting element is located at least partially within said interior.
69. A pacifier kit according to embodiment 68 wherein said stream splitting element has a Y shape defined by a stem and two branches, and said inlet is constituted by said stem and the two outlets are constituted by said branches.
70. A pacifier kit according to any one of embodiments 64-69 wherein said distal portion of said nasal spray add-on device comprises alignment ridges shaped to align said bottle outlet with said inlet.
71. A pacifier kit according to embodiment 70 when dependent on embodiment 68 wherein said alignment ridges are at least partially disposed within said interior.
72. A pacifier kit according to embodiment 66, wherein said fingerhold is constituted by a ridge on an exterior of the distal bottle engaging portion.
73. A pacifier kit according to embodiment 64 wherein said proximal pacifier engaging portion comprises a central aperture configured to receive the add-on accommodating portion of a pacifier.
74. A pacifier kit according to any one of embodiments 63-73, wherein said aromatherapy add-on device comprises;
   at least one recess for an olfactory-active substance to be deposited therewithin, said at least once recess comprising an absorbent material configured to retain and slowly release the add-on the olfactory-active substance, said absorbent material being open to the exterior of the device along at least the majority of the recess; and
   a pacifier engaging portion having such shape and dimensions as to detachably fixable to the add-on receiving portion.
75. A pacifier kit according to embodiment 74 wherein said at least one recess of the aromatherapy add-on device comprises at least one channel.
76. A pacifier kit according to embodiment 75 wherein said at least one channel of the at least one recess of the aromatherapy add-on device is an annular channel extending along an outer surface of the add-on device.
77. A pacifier kit according to embodiment 74 wherein said absorbent material of the aromatherapy add-on device is wood. 78. A pacifier kit according to embodiment 74 wherein said aromatherapy add-on device further comprises a central pacifier engaging aperture having said pacifier engaging portion and configured to be received within the add-on receiving portion of the pacifier.
79. A pacifier kit according to embodiment 74, wherein said aromatherapy add-on device has proximal and distal faces, said proximal face being configured to face the pacifier and said distal face having said recess.
80. A pacifier kit according to embodiment 74, wherein said aromatherapy add-on device is ring shaped.
81. A pacifier kit according to embodiment 74, wherein said aromatherapy add-on device comprises an absorbing element made of said absorbing material and comprising said recess, and an adaptor comprising said pacifier engaging portion and configured for securely holding said absorbing element.
82. A nasal spray add-on device configured for use with a nasal spray bottle having an outlet and containing a fluid to be ejected into nostrils of an infant using said nasal spray add-on device, said nasal spray add-on device comprising;
   an upper portion comprising two nasal ports, said nasal ports being so disposed on two sides of a vertical plane of symmetry and having such a length as to allow them to be insertable into the nostrils of an infant, and
   a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.
83. A nasal spray add-on device according to embodiment 82, wherein said spray bottle comprises a pump, optionally provided with a spring-loaded mechanism.
84. A nasal spray add-on device according to embodiment 83, wherein said nasal spray add-on device further comprises at least one fingerhold configured to be at least partially gripped by the user when actuating said spring-loaded mechanism.
85. A nasal spray add-on device according to embodiment 82 wherein said distal bottle engaging portion comprises a stream splitting element comprising said inlet and two outlets in fluid communication with said two nasal ports.
86. A nasal spray add-on device according to embodiment 85 wherein said distal bottle engaging portion has an interior and the stream splitting element is located at least partially within said interior.
87. A nasal spray add-on device according to embodiment 86 wherein said stream splitting element has a Y shape defined by a stem and two branches, and said inlet is constituted by said stem and the two outlets are constituted by said branches.
88. A nasal spray add-on device according to any one of embodiments 82-87, wherein said distal portion of said nasal spray add-on device comprises at least one alignment ridge shaped to align said bottle outlet with said inlet.
89. A nasal spray add-on device according to embodiment 88 when dependent on embodiment 86 wherein said at least one alignment ridge is at least partially disposed within said interior.
90. A nasal spray add-on device according to embodiment 84, wherein said fingerhold is constituted by at least one gripping ridge on an exterior surface of the distal bottle engaging portion.
91. A nasal spray add-on device according to any one of embodiments 82-90, being further configured for use with a pacifier having an add-on accommodating portion, said nasal spray add-on device comprising a proximal pacifier engaging portion having such shape and dimensions as to be detachably attachable to said add-on accommodating portion of the pacifier.
92. A nasal spray add-on device according to embodiment 92, wherein said proximal pacifier engaging portion comprises a central aperture configured to receive the add-on accommodating portion of a pacifier.
93. A pacifier kit according to any one of embodiments 63-81 further comprising an add-on device according to any one of embodiments 82-90.
94. A pacifier kit according to any one of embodiments 63-81 or embodiment 93, further comprising an oral medicine delivery pacifier, said oral medicine delivery pacifier comprising a nipple, a shield having a first and a second side, and a fluid compartment;
   said nipple being formed on said first side of said shield, and said nipple having an interior lumen;
   said fluid compartment being detachably attachable to the distal side of said shield, or optionally, uniformly formed therewith, and having a reservoir in fluid communication with the interior channel of the nipple, said fluid compartment comprising an outer flexible cap which can be pressed inwards into the fluid compartment to force liquid from the reservoir through the interior channel of the nipple.
95. A method for preparing a nasal spray bottle having an outlet for use in administration of a nasal spray into the nostrils of an infant, comprising providing a nasal spray add-on device having two nasal ports and a bottle engaging portion, and connecting said outlet of said nasal spray bottle to said nasal spray add-on device so as to enable ejecting nasal spray from said nasal spray bottle into said two nasal ports.
96. A method according to embodiment 95, wherein the nasal spray add-on device further comprises;
   an upper portion comprising two nasal ports, said nasal ports being on two sides of a vertical plane of symmetry, so as to be insertable into the nostrils of an infant, and
   a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.
97. A method according to embodiment 96, wherein said spray bottle comprises a pump, optionally provided with a spring-loaded mechanism.
98. A method according to embodiment 97, wherein said ejecting nasal spray from said nasal spay bottle comprises actuating said spring-loaded mechanism.
99. A method according embodiment 98, wherein said nasal spray add-on device further comprises at least one fingerhold configured to be at least partially gripped by the user when actuating said spring-loaded mechanism.
100. A method according to embodiment 96, wherein said distal bottle engaging portion comprises a stream splitting element comprising said inlet and two outlets in fluid communication with said two nasal ports.
101. A method according to embodiment 100, wherein said distal bottle engaging portion has an interior and the stream splitting element is located at least partially within said interior.
102. A method according to embodiment 101 wherein said stream splitting element has a Y shape defined by a stem and two branches, and said inlet is constituted by said stem and the two outlets are constituted by said branches.
103. A method according to any one of embodiments 96-102, wherein said distal portion of said nasal spray add-on device comprises at least one alignment ridge shaped to align said bottle outlet with said inlet.
104. A method according to embodiment 103, when dependent on embodiment 101 wherein said at least one alignment ridge is at least partially disposed within said interior.
105. A method according to embodiment 99, wherein said fingerhold is constituted by a gripping ridge on an exterior surface of the distal bottle engaging portion.
106. A method according to embodiment 97, wherein the nasal spray bottle further comprises an actuator attached to the outlet thereof, and the method further comprises removing said actuator, so that the outlet can be connected to the nasal spray device.
107. A method according to any one of embodiments 94-106, wherein the method further comprises; providing a pacifier having an add-on accommodating portion, and attaching said nasal spray add-on device to said add-on accommodating portion of the pacifier.
108. A method according to embodiment 107, wherein said proximal pacifier engaging portion comprises a central aperture configured to receive the add-on accommodating portion of a pacifier.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 schematically illustrates a schematic side view of a pacifier having an add-on accommodating portion;

FIG. 2A illustrates a schematic side view of an add-on nasal treatment device, which can be assembled with a pacifier of the kind shown in FIG. 1;

FIG. 2B illustrates a schematic side view of an add-on nasal treatment device having a portion for engaging nostrils of an infant and a portion for engaging an external device, which can be assembled with a pacifier of the kind shown in FIG. 1;

FIG. 3A illustrates a perspective view of an exemplary add-on receiving portion and a pacifier engaging portion which can couple together, according to one example of the presently disclosed subject matter;

FIG. 3B illustrates a pacifier having an add-on accommodating portion, according to one example of the presently disclosed subject matter;

FIG. 3C illustrates an exploded view of an exemplary nasal spray add-on device attached to a pacifier engaging portion, according to one example of the presently disclosed subject matter;

FIG. 4A illustrates a schematic exploded side view of the pacifier illustrated in FIG. 1 attached to an aromatherapy add-on device;

FIG. 4B illustrates a schematic exploded side view of a pacifier kit, comprising the pacifier illustrated in FIG. 1 and a nasal spray add-on device;

FIG. 4C illustrates a pacifier kit comprising the pacifier of the kind shown in FIG. 1, the aromatherapy add-on device of FIG. 4A, and the nasal spray add-on device of FIG. 4B;

FIG. 5 illustrates an exploded perspective view of an aromatherapy add-on device, according to one example of the presently disclosed subject matter;

FIG. 6A illustrates a front view of a nasal spray add-on device according to one example of the presently disclosed subject matter;

FIG. 6B illustrates an exploded view of the nasal spray add-on device of FIG. 6A according to one example of the presently disclosed subject matter;

FIG. 6C illustrates a bottom view of the nasal spray add-on device of FIG. 6A according to one example of the presently disclosed subject matter;

FIG. 6D illustrates a cross-sectional view of a portion of the nasal spray add-on device illustrated in FIGS. 6A-6C, that is securely attached to an exemplary nasal spray bottle;

FIG. 7 illustrates a perspective view of a nasal spray add-on device according to one example of the presently disclosed subject matter;

FIG. 8A illustrates a pacifier kit comprising the pacifier of FIG. 3B, the aromatherapy add-on device of FIG. 5, and the nasal spray add-on device of FIGS. 6A-6C; and FIG. 8B illustrates an oral medicine delivery pacifier that can constitute a part of the kit shown in FIG. 8A.

### DETAILED DESCRIPTION OF EMBODIMENTS

The presently disclosed subject matter relates to add-on devices that can be used for delivering nasal and/or oral treatment substances to infants. Such add-on devices can be add-ons for pacifiers and/or other treatment devices. When at least one add-on device of the presently disclosed subject matter is used with a pacifier, the pacifier is configured to detachably receive the at least one add-on device. When at least two add-on devices of the presently disclosed subject matter are used with the pacifier, the pacifier is configured to detachably receive each one of the at least two add-on devices. The at least two add-on devices can be directed for different nasal treatments.

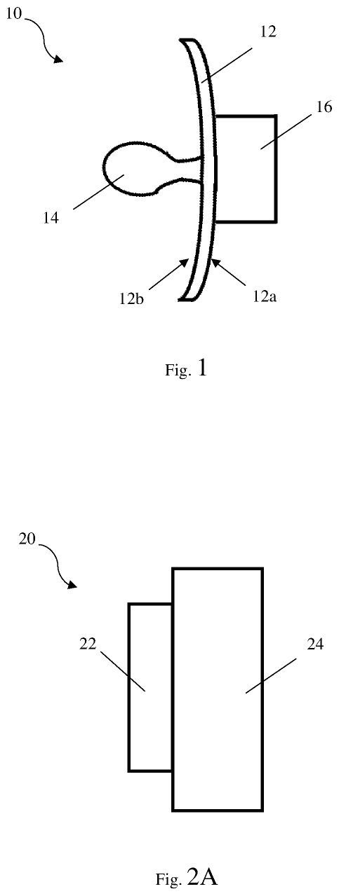

One example of such a pacifier is schematically shown in FIG. 1, where the pacifier is designated as 10, and comprises a mouth plate 12 having a proximal side 12a and a distal side 12b. A nipple 14 is formed on the proximal side 12a of the mouth plate 12 for an infant to suck on, and an add-on receiving portion 16 is formed on the distal side 12b of the mouth plate 12. The add-on receiving portion 16 is configured to receive the at least one add-on device and/or each one of the at least two add-on devices.

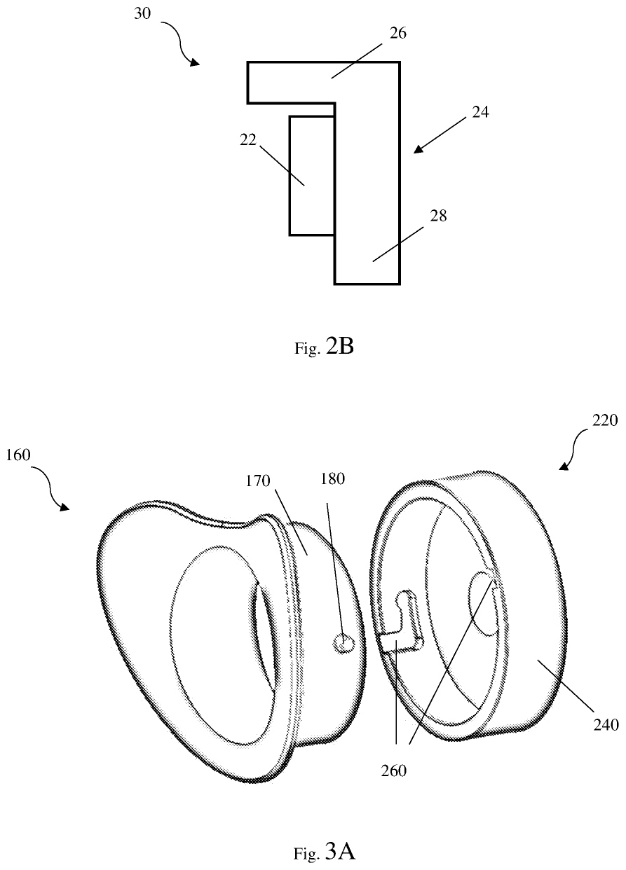

Each one of the at least two add-on devices according to the presently disclosed subject matter comprise a nasal treatment unit and a pacifier engaging portion configured to fixedly and detachably connect to the add-on receiving portion of a pacifier 10. The pacifier engaging portion and the nasal treatment unit can be part of a unitary body or can be separate components that can permanently or detachably connect to each other. Certain nasal treatments may require the introduction of nasal ports into the nostrils of an infant, and certain nasal treatments may not. The nasal treatment unit can provide a means of receiving a medium or can contain the treatment within itself. Two examples of such nasal treatment add-on devices are schematically shown in FIGS. 2A and 2B.

FIG. 2A illustrates a schematic add-on nasal treatment device 20, which can be assembled with a pacifier 10 of the kind shown in FIG. 1. The add-on nasal treatment device 20 comprises a pacifier engaging portion designated as 22, and a nasal treatment unit designated as 24. The pacifier engaging portion 22 is configured to engage the add-on receiving portion 16 of the pacifier 10. FIG. 2B illustrates a schematic add-on nasal treatment device 30, which comprises the features of the add-on nasal treatment device 20 shown in FIG. 2A, and further details a portion for engaging nostrils of an infant, designated as 26, and a portion for engaging an external device, designated as 28.

The add on receiving portion of the pacifier 10 and the pacifier engaging portion of each of the at least two add-on devices can be any appropriate engageable couple. For example, the couple locking mechanism can be any one of a snap-fit, a press fit, or a press and twist fit. The at least two add-on devices can comprise identical pacifier engaging portions, or different pacifier engaging portions that can each couple to the same add-on receiving portion of the pacifier 10.

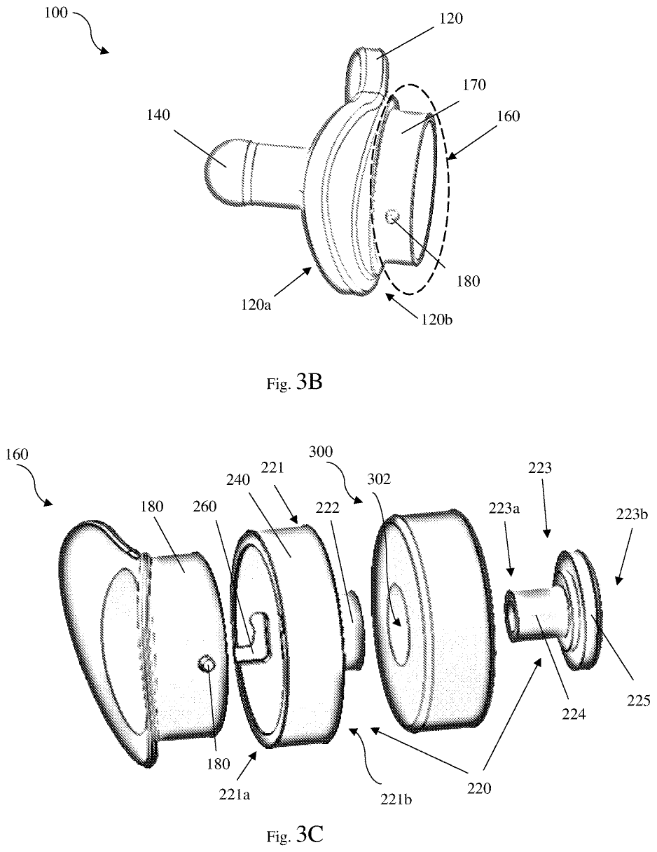

FIG. 3A illustrates an exemplary add-on receiving portion designated as 160 and a pacifier engaging portion designated as 220, which can couple together via a press and twist connection. The add-on receiving portion 160 comprises a ring 170 and two pins 180 diametrically opposed from each other on the exterior of the ring 170. The pacifier engaging portion 220 comprises a cylindrical shell 240 that can envelop the ring 170 of the add-on receiving portion 160. The interior of the cylindrical shell 240 has two L-shaped grooves 260 into which the pins 180 of the add-on receiving portion 160 can be inserted and locked into. The add-on receiving portion of a pacifier according to the presently disclosed subject matter can be a part of or integrally formed with the mouth plate of the pacifier.

FIG. 3B illustrates a pacifier designated as 100, which comprises a mouth plate 120 having a proximal side 120a, and a distal side 120b. A nipple 140 is rigidly attached to and extends from the proximal side 120a. The add-on receiving portion 160 illustrated in FIG. 3A is integrally formed with the distal side 120b of the mouth plate and comprises a ring 170 with two pins 180 on the exterior thereof.

The pacifier engaging portion of an add-on device according to the presently disclosed subject matter may be a unitary body or may comprise an assembly of pieces. For example, in one embodiment the pacifier engaging portion can be unitarily formed with the nasal treatment unit. In another embodiment, the pacifier engaging portion can comprise a first piece having means for coupling with the add-on receiving portion of a pacifier, and a second piece configured to lock the nasal treatment unit to the first piece. The second piece can connect to the nasal treatment unit permanently or detachably, via any suitable means, such as adhesives, a press-fit mechanism or a snap-fit mechanism.

FIG. 3C illustrates an exploded view of the add-on receiving portion 160 of a pacifier, and an exemplary add-on device 300 according to the presently disclosed subject matter. The pacifier engaging portion 220 of the add-on device comprises a first piece 221 having means for coupling with the add-on receiving portion 160 of the pacifier, and a second piece 223 having means for locking the nasal treatment unit 200 to the first piece. The first piece has a proximal side 221a and a distal side 221b. The proximal side 221a is shaped and dimensioned to couple with the add-on receiving portion 160, and the distal side 221b comprises a cylindrical socket 222. The second piece 223 has a distal end 223b comprising a circular plate 224 and a proximal end 223a comprising a cylindrical plug 225. The circular plate can fit into a corresponding groove 301 (not seen) on the nasal treatment unit, and when assembled, the cylindrical plug 225 can extend through a hole 302 in the nasal treatment unit 200 to be tightly received in the cylindrical socket 222 of the first piece 221, thus locking the nasal treatment unit 200 to the pacifier engaging portion 220.

Situating an add-on device according to the presently disclosed subject matter on a pacifier harnesses the dependency infants tend to have on pacifiers and affords multiple advantages for providing nasal treatments to infants. The close proximity of the nasal treatment unit to the nostrils of an infant, and the fixed attachment of the add-on device to the pacifier allow for easy and prolonged access to the nostrils of the infant. Additionally, when sucking on a pacifier an infant can only breath through their nose, thus providing an opportune time to deliver treatment into their nostrils. In the present description, examples of such add-on devices are an aromatherapy providing add-on device and a nasal spray providing add-on device. However, the add-on devices can also be, for example, a combined structure wherein one portion thereof provides aromatherapy and another provides nasal spray, or a water pump having one nasal port to provide nasal irrigation. An aromatherapy add-on device which can be used with a pacifier according to the presently disclosed subject matter comprises, in addition to the pacifier engaging portion, a nasal treatment unit having means for dispensing olfactory materials such as essential oils, infusions, or other aroma compounds to the surrounding air. The aromatherapy nasal treatment unit can be used to provide aromatherapy to an infant by depositing olfactory materials therein, attaching the aromatherapy add-on device to the pacifier, and providing the pacifier to an infant while the olfactory material is slowly released to the surrounding air for the infant to breathe in.

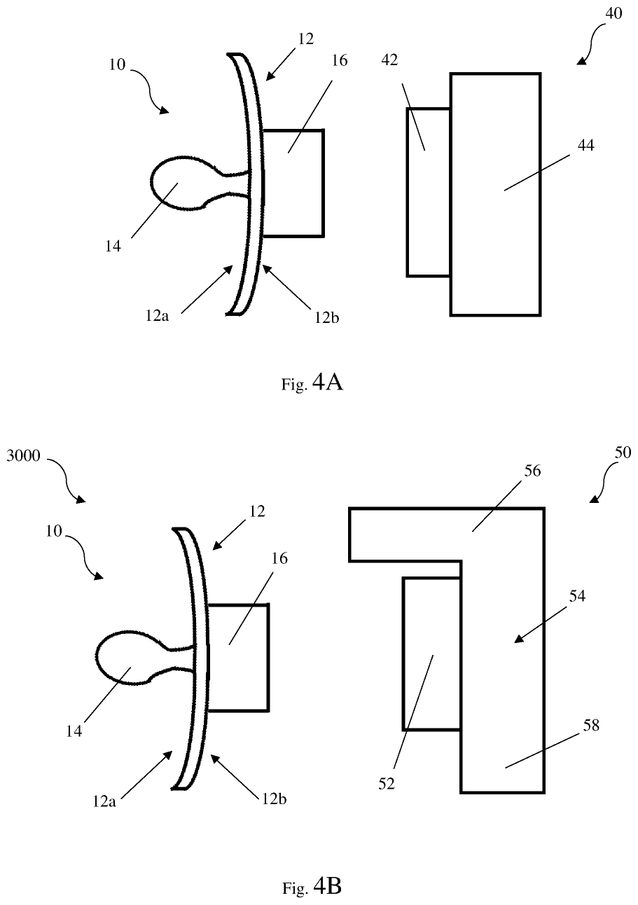

FIG. 4A shows a schematic exploded view of an aromatherapy add-on device designated as 40 that is connected to the pacifier 10. The aromatherapy add-on device 40 comprises a pacifier engaging portion 42 that is shaped and dimensioned to attached to the add-on receiving portion 16 of the pacifier 10 detachably and fixedly. The aromatherapy add-on device 40 additionally comprises a nasal treatment unit 44 configured to receive and slowly release olfactory materials. According to another example, a nasal spray add-on device which can be used with a pacifier according to the presently disclosed subject matter comprises, in addition to the pacifier engaging portion, a nasal treatment unit having means for providing nasal spray to an infant. According to one example of the presently disclosed subject matter, the nasal spray add-on device and the pacifier can constitute a kit. The nasal spray add-on device is configured to be used in conjunction with a nasal spray bottle, to direct spray from the outlet of the nasal spray bottle through the nasal spray add-on device to the nostrils of an infant. To this end, the nasal treatment unit comprises a bottle engaging portion, and a nostril engaging portion. In use, a user first attaches the nasal spray add-on device to the pacifier, and then provides the pacifier to an infant. The user then aligns the spray bottle with the bottle engaging portion and ejects spray from the nasal spray bottle through the nasal nostril engaging portion and into the nostrils of the infant. The nasal spray provided can be a saline solution.

FIG. 4B shows a schematic exploded view of a nasal spray add-on device designated as 50, that is connected to the pacifier 10, which together constitute a kit 3000. The nasal spray add-on device 50 can attach to the add-on receiving portion 16 of the pacifier 10 fixedly and detachably via a pacifier engaging portion 52 thereof. The nasal spray add-on device 50 comprises a nasal treatment unit that 54 has a bottle engaging portion 58 that is configured to interface with a nasal spray bottle.

According to another example of the presently disclosed subject matter, the pacifier, the nasal spray add-on device and at least one additional add-on device according to the presently disclosed subject matter can together constitute a pacifier kit. Such a pacifier kit can afford multiple benefits. Throughout infancy an infant may need different nasal treatments, and in addition to requiring less storage space than a plurality of separate devices, such a pacifier kit will provide convenience. In some cases, it is desirable to provide an infant with different nasal treatments in succession. For example, to treat nasal congestion a saline nasal spray may be used to clean the nasal cavity, followed by aromatherapy to keep the nasal passageways open. Such a pacifier kit will provide necessary tools to tend to such cases. Additionally, it is common for infants to require an adjustment period when adapting to a new pacifier. The at least two add-on nasal treatment devices can be attached to a pacifier according to the presently disclosed subject matter either before providing the pacifier to an infant, or while the infant is already sucking on the pacifier. Since the at least two add-on devices are configured to interchangeably attach to the same pacifier, the infant will not require an adjustment period between different nasal treatments. Such a pacifier kit can also include external devices or materials that are to be used in conjunction with the add-on devices. For example, a pacifier kit may include olfactory materials to be used with an aromatherapy providing add-on device, or a nasal spray bottle to be used with a nasal spray add-on device.

The pacifier 10 and the aromatherapy and nasal spray add-on devices 40, 50 schematically shown in FIGS. 4A and 4B can constitute a pacifier kit designated as 1000. FIG. 4C schematically illustrates the pacifier kit 1000 comprising the pacifier 10 having an add-on receiving portion 16, the aromatherapy add-on device 40, and the nasal spray add-on device 50. The respective pacifier engaging portions 42, 52 of the aromatherapy and nasal spray add-on devices 40, 50 are each configured to fixedly and detachably attach to the add-on receiving portion 16 of the pacifier 10. An aromatherapy add-on device according to the presently disclosed subject matter comprises a pacifier engaging portion and a nasal treatment unit having means for dispensing olfactory materials to the surrounding air. The nasal treatment unit can be in the form of a solid ring or block which has at least one recess for an olfactory-active substance to be deposited therewithin. The at least one recess can be in the form of a groove, a pattern, or a channel, in which the deposited olfactory material can be contained and dispersed. The at least one recess can comprise an absorbent material configured to retain and slowly release the olfactory-active substance. The absorbent material can be a porous material such as clay or wood. In order to allow the olfactory material to diffuse into the air surrounding the pacifier, the absorbent material can be open to the exterior of the device along at least the majority of the recess. The recess can be oriented facing towards or away from the pacifier engaging portion.

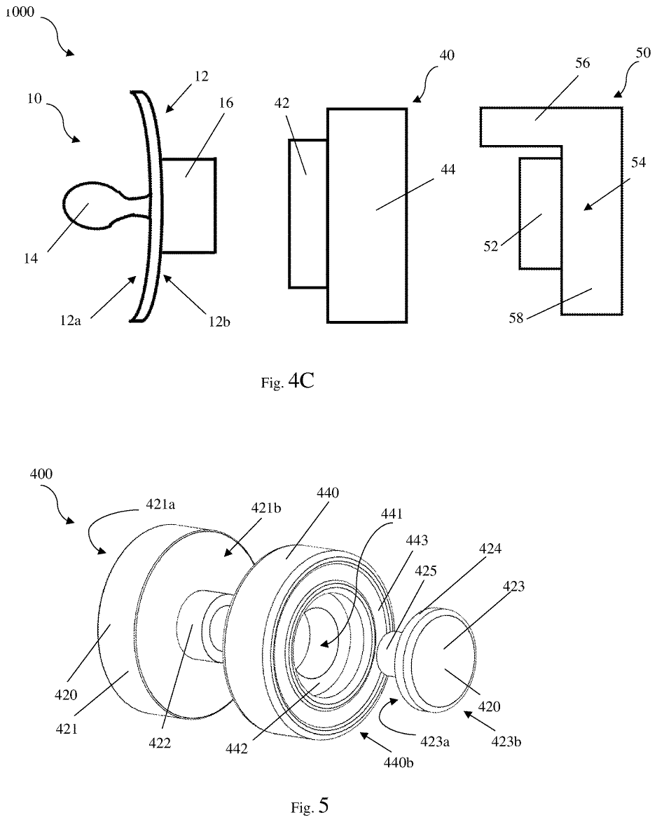

FIG. 5 illustrates a perspective exploded view of a particular example of an aromatherapy add-on device 400 according to the presently disclosed subject matter. The aromatherapy add-on device 400 comprises a nasal treatment unit designated as 440, and a pacifier engaging portion designated as 420 that is configured to be coupleable to the pacifier 100 illustrated in FIG. 3B, and to the nasal treatment unit 440. The pacifier engaging portion 420 comprises a first piece 421 and second piece 423. The first piece 421 has a proximal side 421a and a distal side 421b. The proximal side 421a is shaped and dimensioned in the same way as the proximal side 221a of the first piece 221 of the pacifier engaging portion 220 illustrated in FIG. 3C is, in order to be coupleable with the add-on receiving portion 160 illustrated in FIGS. 3B and 3C. The distal side 421b comprises a cylindrical socket 422. The second piece 423 has a distal end 423b comprising a circular plate 424, and a proximal end 423a comprising a cylindrical plug 425. The nasal treatment unit 440 is in the form of a solid ring with an aperture 441 in its center, and a groove 442 on its distal side 440b. When the aromatherapy add-on device 400 is assembled, the circular plate 424 is received in the groove 442, and the cylindrical plug 425 is inserted through the aperture 441 to be lockingly received by the cylindrical socket 422. The nasal treatment unit 440 comprises a channel 443 on its distal side 440b into which olfactory material can be deposited and dispersed. The channel 443 is made from wood and is open to the exterior of the aromatherapy add-on device 400 along its entirety.

A nasal spray add-on device according to the presently disclosed subject matter comprises a pacifier engaging portion and a nasal treatment unit having means for providing nasal spray to an infant. The pacifier engaging portion is shaped and dimensioned so as to be coupleable to a pacifier having an add-on accommodating portion, and the nasal treatment unit is configured for use with a nasal spray bottle having an outlet and containing a fluid to be ejected into the nostrils of an infant. Such a nasal spray add-on device and pacifier can be a component of a pacifier kit or can constitute an independent device.

The nasal treatment unit can have an upper portion comprising at least one nasal port having such a length to allow it to be insertable into a nostril of an infant when said pacifier is in use. The at least one nasal port can be so positioned on the upper portion as to automatically enter a nostril when the infant sucks on the pacifier, or it can be adjustable to be inserted manually. The at least one nasal port may comprise two nasal ports that are disposed on two sides of a vertical plane of symmetry, to align with the nostrils of an infant. Administering nasal spray to an infant may be particularly challenging if the infant is squirming, and it can be quicker and easier to administer nasal spray to both nostrils simultaneously.

The nasal treatment unit can further comprise a distal bottle engaging portion having an inlet configured to securely and detachably attach to the outlet of the nasal spray bottle. One example of such an attachment can be a press-fit, where at least a portion of the bottle's outlet can be inserted into and securely held by the inlet of the device. The inlet can be in fluid communication with said at least one nasal port, so as to guide ejected fluid from said nasal spray bottle through said at least one nasal port. In an embodiment wherein the at least one nasal port is constituted by two nasal sports, the nasal treatment unit may additionally comprise a stream splitting element to direct ejected fluid from the nasal spray bottle outlet to each nasal port simultaneously. The stream splitting element can be a Y-shaped piece shape defined by a stem and two branches, the stem constituting the inlet of the distal bottle engaging portion, and the two branches being in fluid communication with the two nasal ports. The distal bottle engaging portion can have an exterior and an interior, and the stream splitting element can be located at least partially within the interior. The stream splitting element may be integrally or uniformly formed with the nasal treatment unit, or it may be detachable.

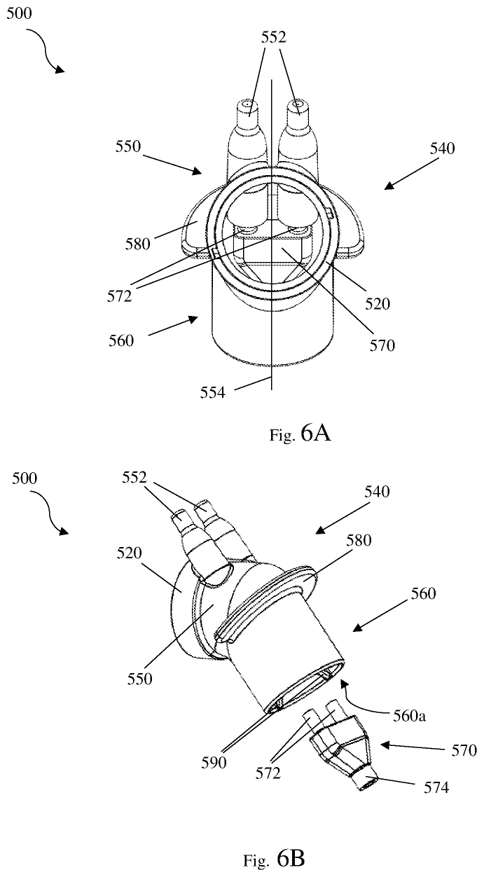

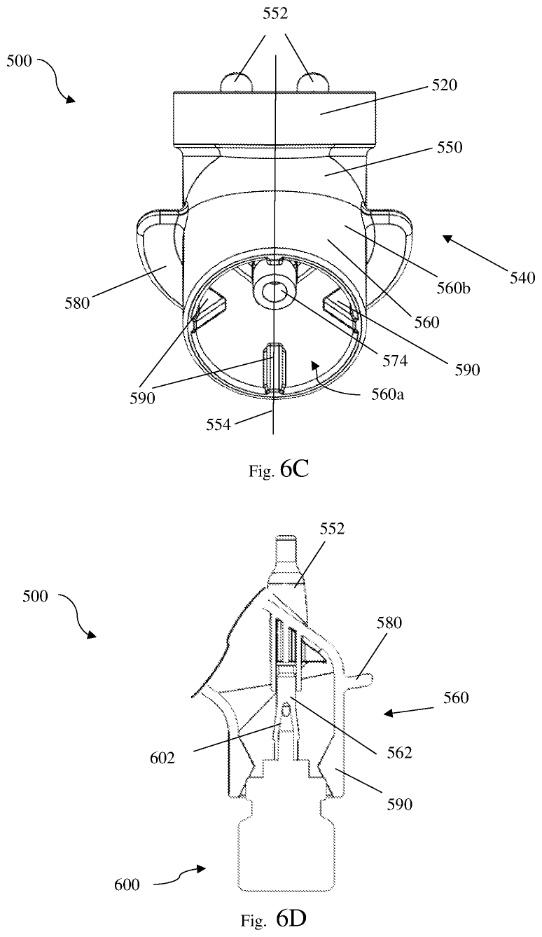

FIGS. 6A and 6B respectively illustrate a front view and a perspective exploded view of a particular example of a nasal spray add-on device 500, according to the presently disclosed subject matter. The nasal spray add-on device 500 comprises a nasal treatment unit designated as 540, and a pacifier engaging portion designated as 520 that is shaped and dimensioned to be coupleable to the add-on receiving portion 160 of the pacifier 100 illustrated in FIG. 3B. The nasal treatment unit 540 has an upper portion 550 and a distal bottle engaging portion 560 having a hollow interior 560a, and an exterior 560b. The upper portion 550 comprises two nasal ports 552 spaced evenly about a vertical plane of symmetry 554. A detachable stream splitting element 570 having a Y-shape defined by two branches 572 and a stem 574 is housed in the hollow interior 560a. The two branches 572 are in fluid communication with the two nasal ports 552 and the stem 574. The stem 574 serves as an inlet that can receive the outlet of a nasal spray bottle.

A nasal treatment unit according to the present example can further comprise a fingerhold for a user to grip when ejecting fluid from the nasal spray bottle. The fingerhold can be in the form of one or more recesses or protrusions formed on the exterior of the nasal treatment unit. For example, the fingerhold can comprise two protrusions spaced evenly about the vertical plane of symmetry on the exterior, or a ridge extending along a portion of the exterior. The nasal treatment unit can additionally comprise a bottle alignment mechanism configured to help the user align the outlet of the bottle with the inlet of the distal bottle engaging portion. The bottle alignment mechanism can be constituted by ridges that are angled towards the inlet so as to guide the outlet of a nasal spray bottle to align with the inlet. The ridges can be located in the hollow interior of the distal bottle engaging portion.

FIG. 6C illustrates a bottom view of the nasal spray add-on device 500 illustrated in FIGS. 6A and 6B. The nasal treatment unit 540 comprises a fingerhold constituted by a ridge 580 that extends along the exterior 560b of the distal bottle engaging portion 560. Alignment ridges 590 are situated on hollow the interior 560a of the distal bottle engaging portion 560 and are angled towards the inlet 574 so as to urge the outlet of a nasal spray bottle to align with the inlet 574.

FIG. 6D illustrates a cross-sectional view of a portion of the nasal spray add-on device 500 illustrated in FIGS. 6A-6C, that is securely attached to an exemplary nasal spray bottle 600 having an outlet 602. The bottle engaging portion 560 comprises an inlet 562. The outlet 602 is securely received within the inlet 562. The upper portion of the nasal spray bottle 600 is in contact with the bottle alignment ridges 590 of the bottle engaging portion 560. The nasal spray add-on device according to the present example can be configured for use with a nasal spray bottle having a spring-loaded outlet. To use a nasal spray bottle having a spring-loaded outlet with the add-on device, a user can first connect the outlet of the nasal spray bottle to the inlet of the distal bottle engaging portion, with the help of the bottle alignment ridges. Some nasal spray bottles comprise a pump having a spring-loaded outlet and an actuator attached thereto. When using such a bottle, the actuator can first be removed, so that the outlet of the pump can be aligned with the inlet of the distal bottle engaging portion. The user can then grip the fingerhold and urge the bottle towards the fingerhold, pressing the bottle outlet against the inlet and causing the spring-loaded outlet to eject nasal spray. The nasal spray will enter the stem of the stream splitting element, travel through the stream splitting element's branches, and exit through the two nasal ports.

In another embodiment of the presently disclosed subject matter, a nasal spray add-on device is constituted solely by a nasal treatment unit having the features, and method of use, as described heretofore with respect to the nasal treatment unit of a nasal spray add-on device. In this case, the add on nasal-spray device does not necessarily have a pacifier engaging portion as described in the corresponding nasal spray add-on device above. The nasal spray add-on device can comprise at least one nasal port. In an embodiment wherein the nasal spray add-on device comprises two nasal ports, the nasal spray add-on device can comprise a stream splitting element that directs nasal spray from the inlet through both nasal ports simultaneously. This embodiment of the nasal spray add-on device can be useful for treating infants, or for treating older children who no longer use a pacifier but are too young to self-administer nasal spray, as it allows for faster treatment compared to a single port sprayer.

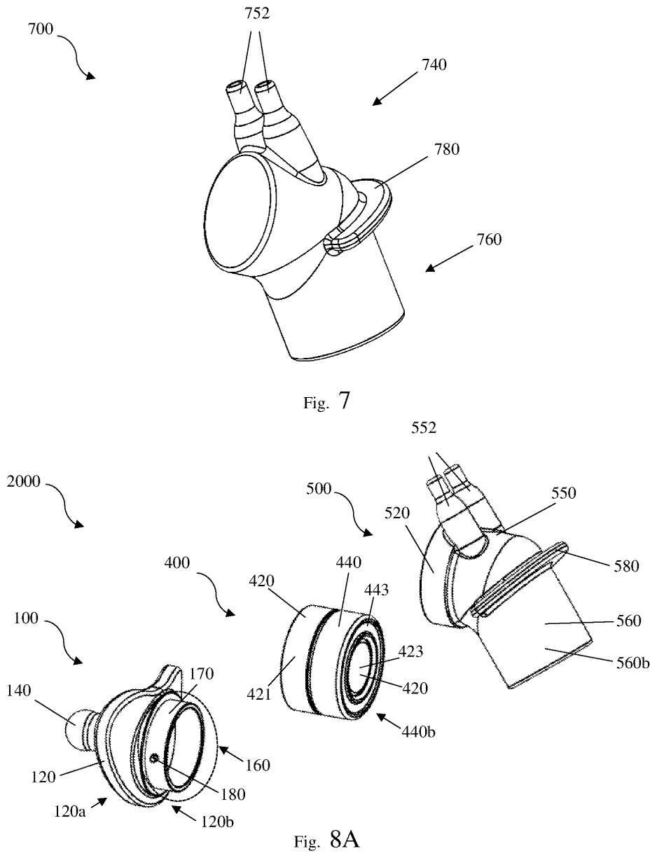

FIG. 7 illustrates a perspective view of a nasal spray add-on device 700. The nasal spray add-on device comprises a distal bottle engaging portion 760 and a nasal treatment unit 740. The nasal treatment unit 740 comprises two nasal ports 752, and a fingerhold constituted by a ridge 780 extends along the exterior 760a of the distal bottle engaging portion 760.

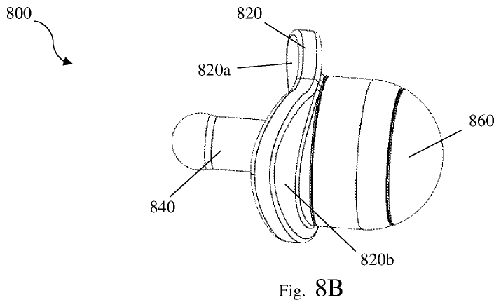

FIG. 8A illustrates a pacifier kit 2000 constituted by the pacifier 100 illustrated in FIG. 3, the aromatherapy add-on device 400 illustrated in FIG. 5, and the nasal spray add-on device 500 illustrated in FIGS. 6A-6C . The pacifier engaging portions 420 and 520 of the aromatherapy and nasal spray add-on devices, respectively, are configured to be interchangeably coupled with the add-on receiving portion 160 of the pacifier 100.

Antibiotics and antipyretic drugs are conventionally administered to infants via a syringe or a spoon. Caregivers frequently encounter challenges when administering such oral medications to infants, since infants often resist taking the medication when presented with a syringe or spoon. Any pacifier kit according to the presently disclosed subject matter may additionally comprise a pacifier that is designed for delivering such oral medicine to an infant. A pacifier of this kind can be a unitary body, or can comprise assemblable portions such as a pacifier having an add-on receiving portion and an oral medicine delivery add-on device. Such an oral medicine delivery pacifier, constituted by a unitary body, is known in the art. For example, U.S. Pat. No. 7,753,886B2 discloses a pacifier that consists of a mouth plate having a distal side and a proximal side. A medicine compartment having a flexible cap is attached to the distal side thereof and a hollow nipple portion is attached to the proximal side thereof. The nipple portion is in fluid communication with the medicine compartment. An infant can suckle on the nipple portion and ingest medicine drawn from the medicine compartment. Additionally, a caregiver can press upon the flexible cap to increase the flow of medicine through the nipple into the mouth of the infant.

FIG. 8B illustrates an oral medicine delivery pacifier designated as 800, which can be included in any pacifier kit according to the presently disclosed subject matter. The oral medicine delivery pacifier 800 has a mouth plate 820 having a proximal side 820a and a distal side 820b. A hollow nipple 840 extends from the proximal side 820a. A flexible medicine compartment 860 is attached to the distal side 820b of the oral medicine delivery pacifier 800 and is in fluid communication with the hollow nipple 840.

1-62. (canceled)

63. A pacifier kit for performing nasal treatment to an infant, said kit comprising at least a pacifier, and at least one nasal spray add-on device directed for a nasal treatment, said pacifier comprising:
a mouth plate having a distal and a proximal side;
a nipple formed on the proximal side; and
an add-on receiving portion formed on the distal side and configured to detachably receive said nasal spray add-on device;
wherein said nasal spray add-on device is configured for use with a nasal spray bottle having an outlet and containing a fluid to be ejected into the nostrils of an infant using said nasal spray add-on device, said nasal spray add-on device comprising:
a proximal pacifier engaging portion having such shape and dimensions as to be detachably attachable to said add-on accommodating portion of the pacifier;
an upper portion comprising two nasal ports, said nasal ports being so disposed on two sides of a vertical plane of symmetry and having such a length as to allow them to be insertable into the nostrils of an infant when said pacifier is in use; and
a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.

64. A pacifier kit according to claim 63, wherein said spray bottle is in the form of a spring-loaded injector comprising a spring-loaded mechanism.

65. A pacifier kit according to claim 63, wherein said proximal pacifier engaging portion comprises a central aperture configured to receive the add-on accommodating portion of a pacifier.

66. A pacifier kit according to claim 63, further comprising at least one additional device directed for a different nasal treatment than said nasal spray add-on device.

67. A pacifier kit according to claim 66, wherein said additional device is constituted by an aromatherapy add-on device, said aromatherapy add-on device comprising:
at least one recess for an olfactory-active substance to be deposited therewithin, said at least once recess comprising an absorbent material configured to retain and slowly release the add-on the olfactory-active substance, said absorbent material being open to the exterior of the device along at least the majority of the recess; and
a pacifier engaging portion having such shape and dimensions as to be detachably fixable to the add-on receiving portion.

68. A pacifier kit according to claim 67, wherein said at least one recess of the aromatherapy add-on device comprises at least one channel.

69. A pacifier kit according to claim 68, wherein said at least one channel of the at least one recess of the aromatherapy add-on device is an annular channel extending along an outer surface of the add-on device.

70. A pacifier kit according to claim 67, wherein said aromatherapy add-on device further comprises a central pacifier engaging aperture having said pacifier engaging portion and configured to be received within the add-on receiving portion of the pacifier.

71. A pacifier kit according to claim 67, wherein said aromatherapy add-on device is ring shaped.

72. A pacifier kit according to claim 67, wherein said aromatherapy add-on device comprises an absorbing element made of said absorbing material and comprising said recess, and an adaptor comprising said pacifier engaging portion and configured for securely holding said absorbing element.

73. A nasal spray add-on device configured for use with a nasal spray bottle having an outlet and containing a fluid to be ejected into nostrils of an infant using said nasal spray add-on device, said nasal spray add-on device further configured for use with a pacifier comprising an add-on receiving portion formed on a distal side thereof and configured to receive said nasal spray add-on device, said nasal spray add-on device comprising:
a proximal pacifier engaging portion having such shape and dimensions as to be detachably attachable to said add-on accommodating portion of said pacifier;
an upper portion comprising two nasal ports, said nasal ports being so disposed on two sides of a vertical plane of symmetry and having such a length as to allow them to be insertable into the nostrils of an infant; and
a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.

74. A nasal spray add-on device according to claim 73, wherein said distal bottle engaging portion comprises a stream splitting element comprising said inlet and two outlets in fluid communication with said two nasal ports.

75. A nasal spray add-on device according to claim 74, wherein said distal bottle engaging portion has an interior and the stream splitting element is located at least partially within said interior.

76. A nasal spray add-on device according to claim 73, wherein said proximal pacifier engaging portion comprises a central aperture configured to receive the add-on accommodating portion of a pacifier.

77. A nasal spray add-on device configured for use with a nasal spray bottle having an outlet and containing a fluid to be ejected into nostrils of an infant using said nasal spray add-on device, said nasal spray add-on device comprising;
an upper portion comprising two nasal ports, said nasal ports being so disposed on two sides of a vertical plane of symmetry and having such a length as to allow them to be insertable into the nostrils of an infant, and
a distal bottle engaging portion having an inlet configured to securely and detachably receive said outlet of the nasal spray bottle, said inlet being in fluid communication with said nasal ports, so as to guide ejected fluid from said nasal spray bottle through said two nasal ports.

78. A nasal spray add-on device according to claim 77, wherein said spray bottle comprises a pump, optionally provided with a spring-loaded mechanism, and wherein said nasal spray add-on device further comprises at least one fingerhold configured to be at least partially gripped by the user when actuating said spring-loaded mechanism.

79. A nasal spray add-on device according to claim 78, wherein said distal bottle engaging portion comprises a stream splitting element comprising said inlet and two outlets in fluid communication with said two nasal ports.

80. A nasal spray add-on device according to claim 79, wherein said distal bottle engaging portion has an interior and the stream splitting element is located at least partially within said interior.

81. A nasal spray add-on device according to claim 80, wherein said stream splitting element has a Y shape defined by a stem and two branches, and said inlet is constituted by said stem and the two outlets are constituted by said branches.

82. A nasal spray add-on device according to claim 77, wherein said distal portion of said nasal spray add-on device comprises at least one alignment ridge shaped to align said bottle outlet with said inlet.
